(19) United States
(12) Patent Application Publication
(10) Pub. No.: US 2002/0049545 A1
(43) Pub. Date: Apr. 25, 2002

(54) Amino acid frame indication system, method for amino acid frame indication, and recording medium

(75) Inventors: Tetsuo Nishikawa, Tokyo (JP); Katsuhiko Murakami, Kokubunji (JP); Takao Isogai, Ami (JP); Keiichi Nagai, Higashiyamato (JP); Koji Hayashi, Toyonaka (JP); Ryoutarou Irie, Kisarazu (JP); Tetsuji Otsuki, Kisarazu (JP)

Correspondence Address: Stanley P. Fisher, Reed Smith Hazel & Thomas LLP, Suite 1400, 3110 Fairview Park Drive, Falls Church, VA, 22042-4503, US

(73) Assignee: Hitachi, Ltd. and Helix Research Institute

(21) Appl. No.: 09/940,664

(22) Filed: Aug. 29, 2001

(30) Foreign Application Priority Data
Oct. 25, 2000 (JP) 2000-325403

(51) Int. Cl. G06F 19/00; G01N 33/48; G01N 33/50

(52) U.S. Cl. 702/19

(57) ABSTRACT

The object of the present invention is to provide an amino acid frame indication system, a method for amino acid frame indication and a recording medium, which can effectively extract a highly reliable amino acid sequence from a cDNA sequence, even in a case where there exists a frame shift error in the cDNA sequence.

It becomes possible to obtain a highly precise amino acid sequence by: expressing the amino acid information of a cDNA sequence obtained by similarity comparison with known amino acid sequences, together with the ORF display of the cDNA sequence on each amino acid frame; effectively detecting a frame shift by displaying both the plausibility of an initiation codon which is information regarding an ORF statistically obtained at the same time, and a coding potential graph; and editing the obtained results.

### DETAILED DESCRIPTION OF THE INVENTION

1. Field of the Invention

The present invention relates to an amino acid frame indication system, a method for amino acid frame indication and a recording medium, which involve analysis of a gene sequence for the purpose of identifying an amino acid sequence encoded by the gene sequence.

2. Prior Art

The development of the Human Genome Project (the Draft Sequence was completed in June, 2000) has brought about a rapid expansion of the range of databases concerning gene sequences as well as an increase in the throughput of sequence determination. EST sequences registered in high volume (partial gene sequences) and draft sequences (low precision arrangements before completion of the genomic sequence) are sequences which are collected with an emphasis on throughput, and so the precision of these sequences is not very high (It is said that about 3% of EST sequences is error). It is required that amino acid sequence information with precision that is as high as possible is extracted from these sequences. Conventionally, for the extraction of amino acid sequence information from a cDNA sequence, an amino acid frame display has generally been used (ORF Finder, hlt ://www.ncbi.nlm.nih.gov/gorf/gorf.html).

The amino acid frame display indicates 3 amino acid sequences obtained by translating by shifting one letter from 5′-end of a cDNA sequence as 3 segments. Where a reverse complementary strand is taken into consideration, 6 amino acid sequences as a whole are displayed as 6 segments. On these segments, each position of initiation and termination codons is displayed differently and a segment which starts at an initiation codon and terminates at a termination codon is identified.

The thus obtained segments are identified as possible open reading frames (ORF), and among them, the longest ORF is identified as an amino acid sequence extracted from the cDNA. Where a frame shift error exists on a cDNA sequence, an ORF is split and displayed over 2 frames by the amino acid frame display. Further, since the border of the split ORF is not clear, an amino acid sequence is, in general, identified with an error of tens of bases. Accordingly, when a frame shift error exists on a cDNA sequence, the frame shift error has previously been identified using similarity information to known amino acid sequences. The most common program to compare a cDNA sequence with an amino acid sequence is BLASTX (Altschul, S.F., et al., Basic local alignment search tool, J. Mol. Biol., 215(3), 403, 1990) which has been developed by the National Center for Biotechnology Information (NCBI), U.S.A. This BLASTX translates a given cDNA sequence into 6 possible amino acid sequences (6 frames), performs a similarity comparison of these sequences with amino acid sequences in a database, and as a result, outputs an alignment between amino acid sequences. When one frame shift error exists on a cDNA sequence, an alignment which should be obtained under normal conditions is split into 2 alignments. Where there is a high similarity as a whole, it is possible, though with considerable effort, to reconstruct the original alignment from the split alignments and identify a frame shift site. Where there is a low similarity as a whole, however, it is difficult to reconstruct the original alignment from the split alignments to identify a frame shift site. As a method of comparing a cDNA sequence with an amino acid sequence in consideration of the occurrence of frame shift errors, a method of obtaining an alignment has been published (Japanese Patent Application Laid-Open (kokai) No. 10-5000). Using this method, even where a frame shift error exists, the only alignment can be obtained and it becomes possible to identify a frame shift site. Nevertheless, even where this method is used, where a similarity is low as a whole, it is difficult to evaluate the reliability of the obtained amino acid sequence. Thus, to extract an amino acid sequence from a cDNA sequence, there are two methods: a method of using an amino acid frame and a method of using similarity information to known amino acid sequences. However, in order to extract a highly reliable amino acid sequence even where a frame shift error exists on a cDNA sequence, the application of either one of these methods is not sufficient.

Object to be Achieved by the Invention

The object to be achieved by the present invention is to provide an amino acid frame indication system, a method for an amino acid frame indication and a recording medium, which are able to effectively extract a highly reliable amino acid sequence from a cDNA sequence, even where a frame shift error exists on the cDNA sequence.

Means to Achieve the Object

The present invention enables effective high-precision performance of the identification and editing of a frame shift error in a sequence, by performing a statistical analysis of a sequence and a similarity analysis with known amino acid sequences on a target gene sequence and displaying the results on an amino acid frame in an integrated manner.

Accordingly, the present invention is directed to the effective extraction of a highly reliable amino acid sequence from a cDNA sequence by a method consisting of the following steps relative to the cDNA sequence:

(1) an analysis step by an initiation codon prediction program, ATGpr,

(2) a coding potential analysis step which is an indicator of coding region plausibility of a DNA sequence on 3 extracted ORFs,

(3) a detection step by a homology detection program against an amino acid sequence database,

(4) a step for displaying the results of the above 3 analyses concurrently with amino acid frame information,

(5) a step for editing the possible portion where a frame shift error would occur, while referring to the above display results, and

(6) a step for storing the above analysis and editing results into a hard disk.

The present invention provides an amino acid frame indication system which comprises: input means for inputting a cDNA sequence; translation means for obtaining 3 amino acid frames translated by shifting one letter per frame along the input cDNA sequence; alignment means for generating an alignment between the input cDNA sequence and a DNA or amino acid sequence in a database to determine from the alignment an amino acid sequence translated from the input cDNA sequence on a basis of similarity information; and display means for displaying as a segment a region of the amino acid sequence determined by the alignment means on the 3 amino acid frames.

Moreover, the present invention provides an amino acid frame indication system which comprises: input means for inputting a cDNA sequence; translation means for obtaining 3 amino acid frames translated by shifting one letter per frame along the input cDNA sequence; codon prediction means for predicting each of initiation and termination codons in the 3 amino acid frames; and display means for displaying an amount or symbol expressing the plausibility of an initiation codon at the initiation codon position as well as displaying the positions of the initiation and termination codons on the 3 amino acid frames.

Furthermore, the present invention provides an amino acid frame indication system which comprises: input means for inputting a cDNA sequence; translation means for obtaining 3 amino acid frames translated by shifting one letter per frame along the input cDNA sequence; codon prediction means for predicting each of initiation and termination codons in the 3 amino acid frames; coding potential calculation means for calculating coding potential showing coding region plausibility in each of the 3 amino acid frames; and display means for displaying the coding potential of the 3 amino acid frames on each frame or in another window as well as displaying the positions of the initiation and termination codons on the 3 amino acid frames.

Further, the present invention provides a method for amino acid frame indication which comprises: an input step for inputting a cDNA sequence; a translation step for obtaining 3 amino acid frames translated by shifting one letter per frame along the input cDNA sequence; an alignment step for generating an alignment between the input cDNA sequence and a DNA or amino acid sequence in a database to determine from the alignment an amino acid sequence translated from the input cDNA sequence on the basis of similarity information; and a display step for displaying as a segment a region of the amino acid sequence determined by the alignment steps on the 3 amino acid frames.

Still further, the present invention provides a method for amino acid frame indication which comprises: an input step for inputting a cDNA sequence; a translation step for obtaining 3 amino acid frames translated by shifting one letter per frame along the input cDNA sequence; a codon prediction step for predicting each of initiation and termination codons in the 3 amino acid frames; and a display step for displaying an amount or symbol expressing the plausibility of an initiation codon at the initiation codon position, as well as displaying the positions of the initiation and termination codons on the 3 amino acid frames.

Moreover, the present invention provides a method for amino acid frame indication which comprises: an input step for inputting a cDNA sequence; a translation step for obtaining 3 amino acid frames translated by shifting one letter per frame along the input cDNA sequence; a codon prediction step for predicting each of initiation and termination codons in the 3 amino acid frames; a coding potential calculation step for calculating coding potential showing coding region plausibility in each of the 3 amino acid frames; and a display step for displaying the coding potential of the 3 amino acid frames on each frame or in another window, as well as displaying the positions of the initiation and termination codons on the 3 amino acid frames.

Further, the present invention provides a computer-readable recording medium on which is recorded a program which allows a computer to function as an amino acid frame indication system which comprises: input means for inputting a cDNA sequence; translation means for obtaining 3 amino acid frames translated by shifting one letter per frame along the input cDNA sequence; alignment means for generating an alignment between the input cDNA sequence and a DNA or amino acid sequence in a database to determine from the alignment an amino acid sequence translated from the input cDNA sequence on the basis of similarity information; and display means for displaying as a segment a region for the amino acid sequence determined by the alignment means on the 3 amino acid frames.

Furthermore, the present invention provides a computer-readable recording medium which records a program to allow a computer to function as an amino acid frame indication system which comprises: input means for inputting a cDNA sequence; translation means for obtaining 3 amino acid frames translated by shifting one letter per frame along the input cDNA sequence; codon prediction means for predicting each of initiation and termination codons in the 3 amino acid frames; and display means for displaying an amount or symbol expressing the plausibility of an initiation codon at the initiation codon position as well as displaying the positions of the initiation and termination codons on the 3 amino acid frames.

Still further, the present invention provides a computer-readable recording medium on which is recorded a program which allows computer to function as an amino acid frame indication system which comprises: input means for inputting a cDNA sequence; translation means for obtaining 3 amino acid frames translated by shifting one letter per frame along the input cDNA sequence; codon prediction means for predicting each of initiation and termination codons in the 3 amino acid frames; coding potential calculation means for calculating coding potential showing the plausibility of coding region in each of the 3 amino acid frames; and display means for displaying the coding potential of the 3 amino acid frames on each frame or in another window, as well as displaying the positions of the initiation and termination codons on the 3 amino acid frames.

### BRIEF DESCRIPTION OF DRAWINGS

FIG. 1 is a figure showing the configuration of an amino acid frame indication system in one embodiment of the present invention.

FIG. 2 shows a figure showing a window transition and the flow of analysis in one embodiment of the present invention.

FIG. 3 is a figure showing a flow chart of sequence analysis.

FIG. 4 is a figure showing an overview of the display window of analysis results.

FIG. 5 is a figure showing a method for displaying similarity information on an amino acid frame (an example of a pre-editing window).

FIG. 6 is a figure showing a method for displaying coding potentials along a cDNA sequence.

FIG. 7 is a figure showing a method for visually displaying information re

FIG. 8 is a figure showing the text display of an alignment and an editing window.

FIG. 9 is a figure showing a method for displaying similarity information on an amino acid frame (an example of a post-editing window).

### DEFINITIONS FOR NUMBER SIGNS

101: User operating the present system

102: cDNA sequence and analysis parameter input window

103: Parameter display and sequence display pane

104: Start button for analysis process

105: Start button for analysis results read and post-analysis process

106: cDNA sequence analysis and display process

107: cDNA sequence analysis results read and display process

108: Analysis results display window

109: Analysis result display and parameter display pane

110: Start button for parameter alteration process

111: Button for opening editing window

112: Start button for analysis results saving process

113: Parameter alteration process

114: cDNA sequence editing window

115: Alignment display pane

116: Post-analysis start button

117: cDNA sequence and analysis results save process

118: Hard disk for storing cDNA sequence and analysis results

201: Process of extracting an ORF from a cDNA sequence

202: cDNA sequence similarity analysis process

203: BLASTX analysis process

204: TRANSQ analysis process

205: Alignment information extraction process

206: cDNA sequence statistical analysis process

207: ATGpr analysis process

208: Coding potential analysis process

209: Amino acid sequence database

301: Analysis results display window

302: Pane for displaying amino acid frames

303: Pane for displaying coding potential

304: Pane for displaying an amino acid alignment

401: cDNA sequence scale

402: Frame 1 obtained by translating a cDNA sequence into an amino acid sequence taking the 1^{st} base from the 5′-end as a starting point,

403: Frame 2 obtained by translating a cDNA sequence into an amino acid sequence taking the 2^{nd} base from the 5′-end as a starting point,

404: Frame 3 obtained by translating a cDNA sequence into an amino acid sequence, taking the 3^{rd} base from the 5′-end as a starting point,

405: Positions showing initiation codons (ATG) on each frame

406: Positions showing termination codons on each frame

407: Longest segment (the longest ORF in a frame) among segments from initiation codons to termination codons (ORF) on each frame

408: Segments straddling each frame which display the amino acid sequence determined by the alignment between a cDNA sequence and an amino acid sequence.

409: Value (the output of ATGpr) showing the plausibility of an ORF initiating from an initiation codon in respect of each initiation codon

501: cDNA sequence scale

502: Coordinate indicating coding potential values in each region along a cDNA sequence

503: Coding potential value of Frame 1

504: Coding potential value of Frame 2

505: Coding potential value of Frame 3

506: Check box for deciding display or non-display of coding potential of Frame 1

507: Check box for deciding display or non-display of coding potential of Frame 2

508: Check box for deciding display or non-display of coding potential of Frame 3

509: Window Size display for coding potential value calculation and input box for altering value

510: Window Size shift value display for coding potential value calculation and input box for altering value

511: Window Size for coding potential value calculation and button for altering shift value for recalculation

601: cDNA sequence scale

602: First alignment between a cDNA sequence and an amino acid sequence

603: Second alignment between a cDNA sequence and an amino acid sequence

604: Third alignment between a cDNA sequence and an amino acid sequence

605: Region wherein Identity≧90% in an alignment between a cDNA sequence and an amino acid sequence

606: Region wherein 90%>Identity>40% in an alignment between a cDNA sequence and an amino acid sequence

607: Region wherein 40%>Identity in an alignment between a cDNA sequence and an amino acid sequence 608: Region which is not aligned in an alignment between a cDNA sequence and an amino acid sequence

609: Region wherein DNA is inserted (where insertion number is multiples of 3) in an alignment between a cDNA sequence and an amino acid sequence

610: Region wherein DNA is deleted (where deletion number is multiples of 3) in an alignment between a cDNA sequence and an amino acid sequence

611: Check box for selecting the first alignment between a cDNA sequence and an amino acid sequence

612: Check box for selecting the second alignment between a cDNA sequence and an amino acid sequence

613: Check box selecting the third alignment between a cDNA sequence and an amino acid sequence

614: Pane of displaying values showing the characteristics of an alignment between a cDNA sequence and an amino acid sequence (Identity, E-value of blastx analysis, length of alignment, length of 5′-end non-aligned DNA side and length of 5′-end non-aligned amino acid side)

615: Information regarding amino acids (ID, definition etc.) in respect of an alignment between a cDNA sequence and an amino acid sequence

701: Alignment display between a cDNA sequence and an amino acid sequence

702: Example of insertion of an a-base into a cDNA sequence

703: Button for determining the editing of a cDNA sequence

704: An alignment between a cDNA sequence and an amino acid sequence, and editing window close button

705: Reset button for editing of a cDNA sequence

801: cDNA sequence scale

802: Frame 1 obtained by translating a cDNA sequence into an amino acid sequence taking the 1^{st} base from the 5′-end as a starting point,

803: Frame 2 obtained by translating a cDNA sequence into an amino acid sequence, taking the 2^{nd} base from the 5′-end as a starting point,

804: Frame 3 obtained by translating a cDNA sequence into an amino acid sequence, taking the 3^{rd} base from the 5′-end as a starting point,

805: Positions of initiation codons (ATG) on each frame

806: Positions of termination codons on each frame

807: Longest segment (longest ORF in the frames) among segments from initiation codons to termination codons (ORF) on each frame

808: Segments straddling frames which display an amino acid sequence determined by the alignment between a cDNA sequence and an amino acid sequence

809: Value (output of ATGpr) showing the plausibility of an ORF initiating from an initiation codon in respect of each initiation codon

Embodiments for Carrying out the Invention

Hereinafter, the preferred embodiments of the present invention are further described, while referring to the attached drawings.

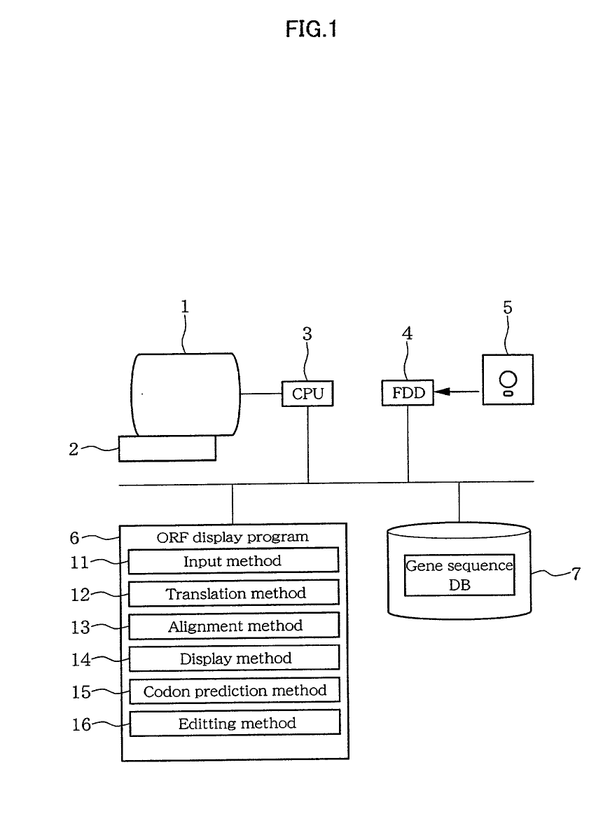

FIG. 1 is a figure showing the configuration of an amino acid frame indication system in one embodiment of the present invention. This embodiment is constituted by display (1), keyboard (2), central processing unit (CPU) (3), floppy disk drive (4) into which floppy disk (5) is inserted, main memory (6) and gene sequence database (7). Stored on main memory (6) is an amino acid frame indication program which realizes an amino acid frame indication system, and the program has functions corresponding to each of input means (11), translation means (12), alignment means (13), display means (14), codon prediction means (15) and editing means (16). This program is executed in CPU (3) in cooperation with display (1), keyboard (2), floppy disk drive (4), main memory (6) and gene sequence database (7).

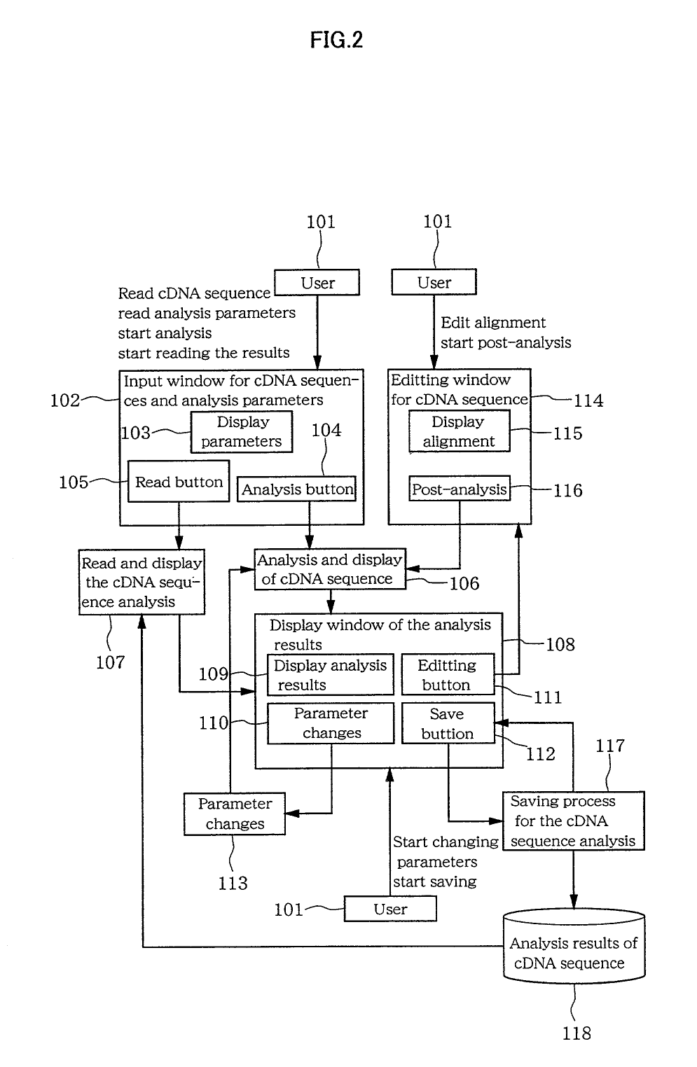

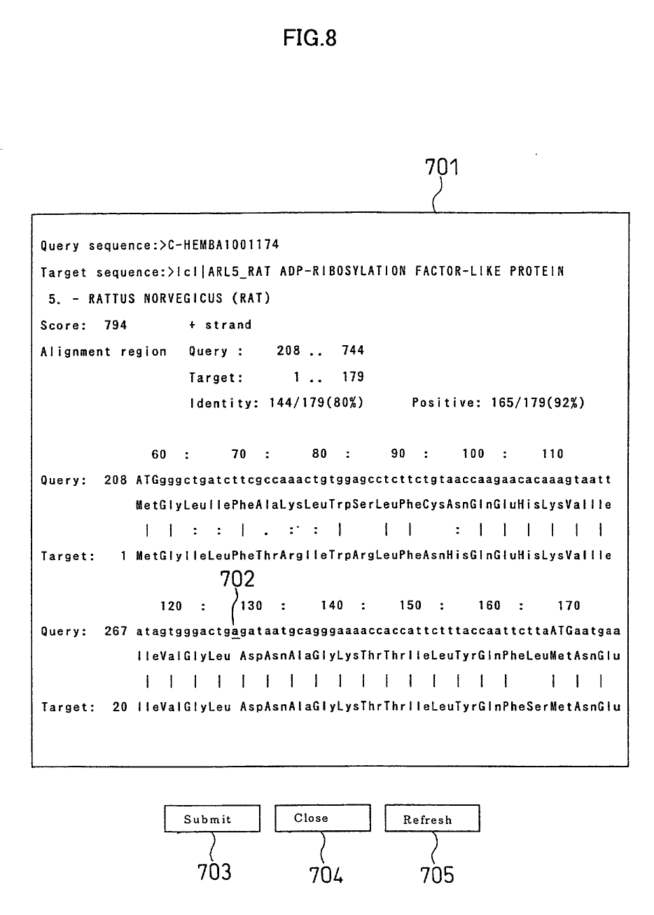

An overview of the system is described using FIG. 2. When the system is booted-up by user (101), cDNA sequence and analysis parameter input window (102) is displayed. Within window (102), an input box for default parameter values and cDNA sequences are displayed in parameter display and sequence display pane (103). User (101) can perform input of cDNA sequences and analysis parameters. Window (102) displays analysis processing start button (104), and analysis and display of cDNA sequence is executed when user (101) pushes this button. Furthermore, window (102) also displays analysis results read button (105) for starting read from hard disk (118) which has cDNA sequence analysis results stored thereon. When user (101) pushes this button, display of cDNA sequence analysis results is executed. Display window (108) (described in detail in FIG. 4) is displayed by cDNA sequence analysis and display process (106) or cDNA sequence analysis results read and display process (107). Analysis results and analysis parameter values are displayed in analysis results and parameters pane (109) within display window (108). Furthermore, display window (108) displays parameter alteration button (110), editing button (111) and save button (112). User (101) is able to alter analysis parameters while viewing analysis results (109) in display window (108) and rerun the analysis. Parameter alteration (113) is started by pushing the parameter alteration button (110). After parameter alteration (113), cDNA sequence analysis and display process (106) is executed again. User (101) is able to edit a cDNA sequence, while viewing analysis results (109) in display window (108). cDNA sequence editing window (114) (described in detail in FIG. 8) is opened by pushing editing button (111). cDNA sequence editing window (114) displays alignment (115) between a cDNA sequence and an amino acid sequence. User (101) is able to directly edit a cDNA sequence in alignment display (115), while referring to analysis results in display window (108). After completion of editing, cDNA sequence analysis and display process (106) can be restarted by pushing post-analysis button (116). The results are displayed in analysis results display window (108) again, so that the effect of editing can be confirmed. User (101) is able to name the cDNA sequence and the analysis results and save them to a hard disk as electronic files. A cDNA sequence and analysis results saving process is started by pushing save button (112), and the cDNA sequence and analysis results are saved to a file in a hard disk (118).

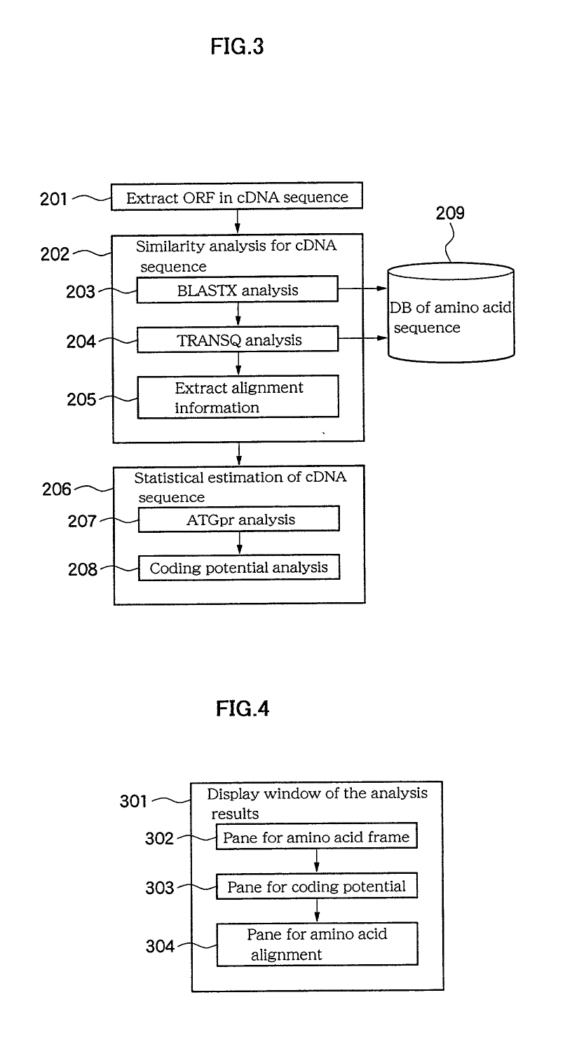

The analysis step of a cDNA sequence is described using FIG. 3. First, according to ORF extraction step (201), ORF information, i.e., an initiation codon, a termination codon and frame information thereof are extracted from the input cDNA sequence. Then, cDNA sequence similarity analysis process (202) is executed. In similarity analysis (202), BLASTX analysis process (203) is executed using amino acid sequence database (209) as a target database. From a hit list obtained by BLASTX, a certain number of database entries are extracted in increasing order of similarity scale, e.g., E-value. Then, TRANSQ analysis process (204) is executed between those amino acid sequences and a cDNA sequence. While translating a cDNA sequence, the TRANSQ takes frame shift in the cDNA sequence into consideration and generates an alignment between the cDNA sequence and an amino acid sequence. On account of this, there can be obtained an amino acid sequence translated from a cDNA sequence, wherein frame shift in the cDNA sequence was taken into consideration. From the obtained alignment, frame shift information and the information of the amino acid sequence translated from the cDNA sequence are extracted by alignment information extraction process (205). In respect of the amino acid sequence translated from the cDNA sequence used herein, one obtained by BLASTX can also be used. Subsequently, cDNA sequence statistical analysis process (206) is executed. First, a score indicating the plausibility of an initiation codon is calculated for each initiation codon ATG contained in a cDNA sequence by ATGpr analysis process (207). The ATGpr calculates a score indicating the plausibility of an initiation codon based on the statistical properties of the cDNA sequence, using a program developed by Helix Research Institute (Salamov, A. A., et al., Assessing Protein Coding Region Integrity in cDNA Sequencing Projects, Bioinformatics, 14, 384, 1998). Then, coding potential analysis is executed by coding potential analysis process (208). In the coding potential analysis process, the plausibility of a coding region is calculated for each frame in a window of a given length sequence in a cDNA sequence, and then the plausibility of a coding region is sequentially calculated, while sliding the window. An indicator showing the plausibility of a coding region is obtained by frequency statistical analysis of a character string consisting of about 6 bases.

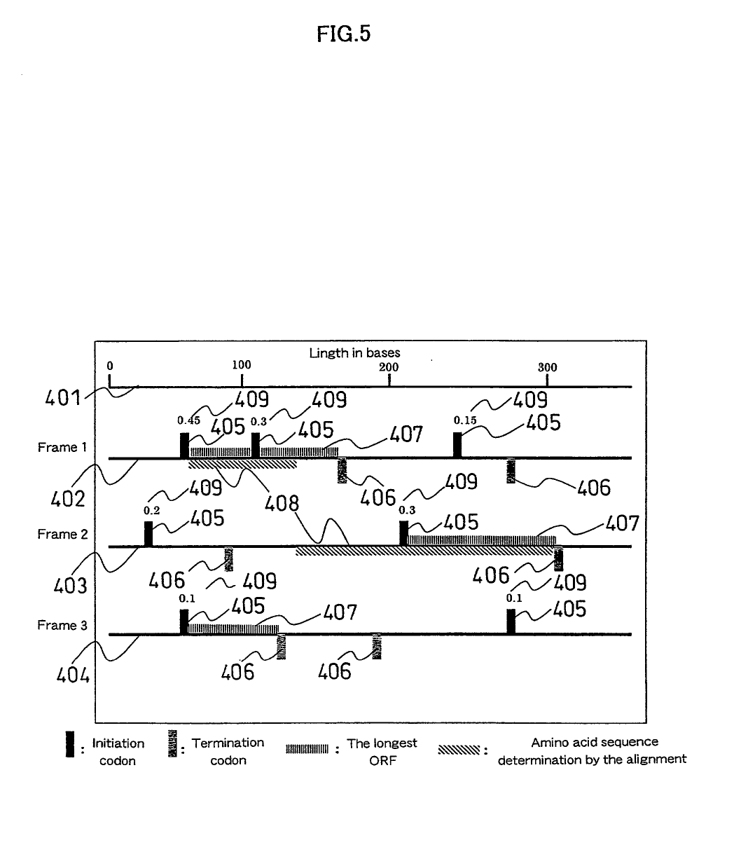

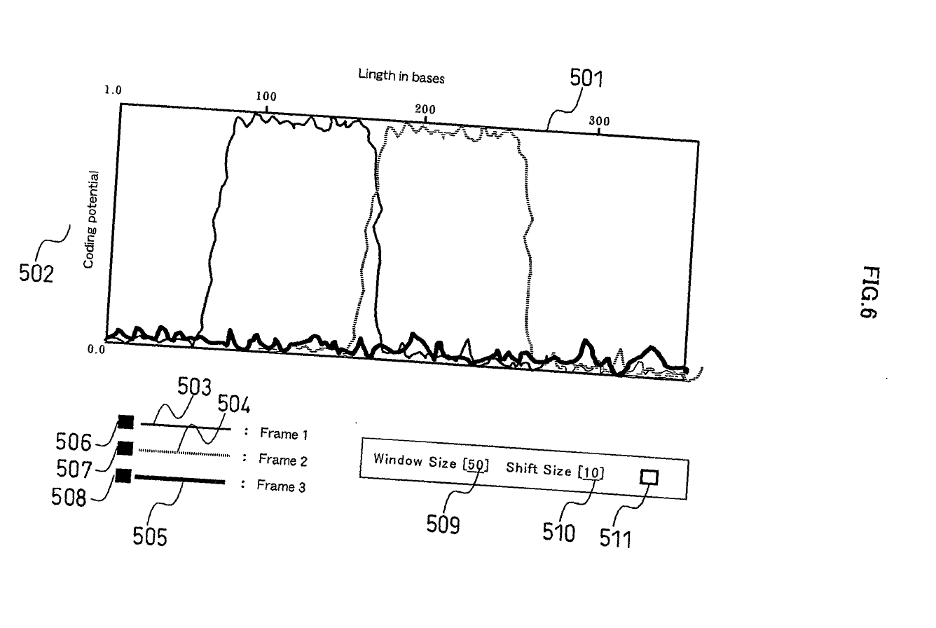

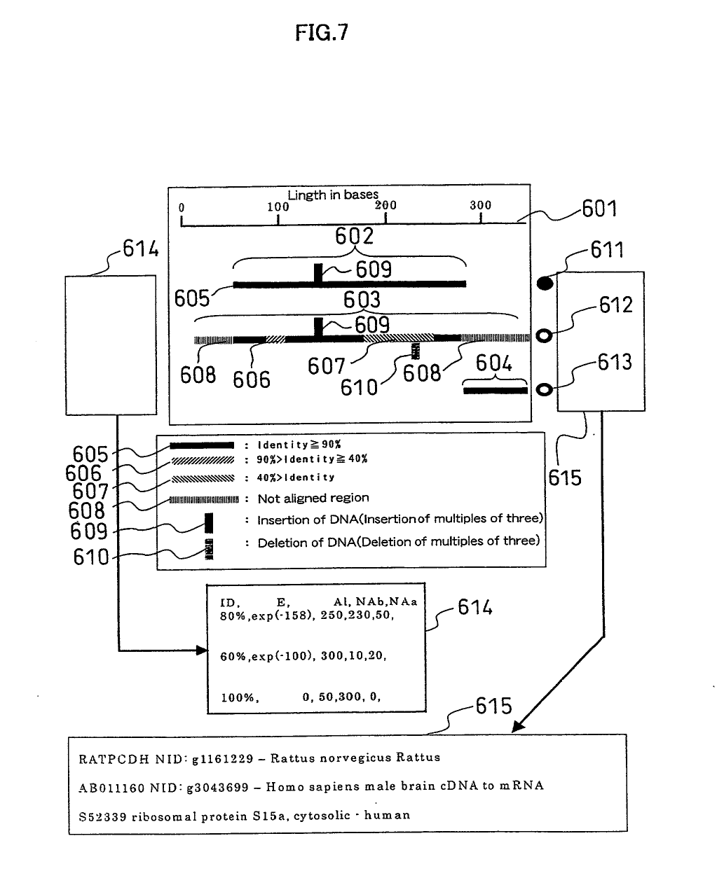

An analysis results display window is described below. FIG. 4 shows an overview of the analysis results display window. Analysis results display window (301) is constituted by amino acid frame display pane (302) (described in detail in FIG. 5), coding potential display pane (303) (described in detail in FIG. 6) and amino acid alignment display pane (304) (described in detail in FIG. 7).

Each pane is described in detail. The amino acid frame display pane (302) displays similarity information as well as amino acid frames. Details are described in FIG. 5. Setting cDNA sequence scale (401) as a coordinate, 3 amino acid sequence frames are displayed. That is to say, the following 3 frames are displayed as segments: frame 1 (402) obtained by translating a cDNA sequence into amino acid sequence taking the 1^{st} base from the 5′-end as a starting point, frame 2 (403) obtained by translating a cDNA sequence into amino acid sequence taking the 2^{nd} base from the 5′-end as a starting point, and frame 3 (404) obtained by translating a cDNA sequence into amino acid sequence taking the 3^{rd} base from the 5′-end as a starting point. On each frame, the position of an initiation codon (ATG) (405) and the position of a termination codon (406) are displayed as bars. Furthermore, the longest segment (407) (the longest ORF in the frames) among segments from initiation codons to termination codons (ORF) on each frame is displayed as a cross line. This is a commonly used display method. Generally deeming the longest ORF from among all frames to be a plausible ORF, the amino acid sequence is set as an object for the following analysis. Where relatively long ORFs exist astride a plurality of frames, a frame shift may exist in regions existing between those ORFs. Herein, a frame shift may exist in a pane between the longest ORFs in frames 1 and 2. With this information alone, however, it is not possible to specify the position where the frame shift exists. Hence, in the present invention, both similarity information to known amino acid sequences and statistical information which a cDNA sequence possesses, are used. As similarity information, the amino acid sequence determined from the alignment between a cDNA sequence and an amino acid sequence is displayed as a segment (408) sitting astride frames. Segment (408) is displayed as being astride frames 1 and 2, and it can be seen that this transition between frames causes a frame shift. As statistical information of a cDNA sequence, the output of ATGpr (409) is displayed near each initiation codon. With this output, the plausibility of ORF starting from each initiation codon is not only displayed as a length but also a value.

In coding potential indication pane (303), coding potential information is displayed along a cDNA sequence. The details are described using FIG. 6. Setting cDNA sequence scale (505) as a horizontal axis, a coding potential is displayed on coordinate (502). As coding potentials, frame 1 coding potential (503), frame 2 coding potential (504), and frame 3 coding potential (505) are displayed. Whether or not coding potential is to be displayed for frames 1, 2 and 3 can be determined with check box (506), check box (507) and check box (508). In respect of calculating coding potential, as stated above, coding region plausibility is calculated for each frame in a certain-length sequence window of a cDNA sequence, and plausibility is sequentially calculated, while sliding the window. The indicator of coding region plausibility can be obtained by frequency statistical analysis of a character string consisting of about 6 bases. As shown in FIG. 6, a region having a high coding potential value swithces from frame 1 to frame 2 around the 130 base length point. This suggests the existence of a frame shift at around 130 base length. Thus, it is possible to estimate the existence and position of a frame shift by observing the transition of coding potential value between frames. When coding potential is calculated, both window size and shift value are displayed in box (509) and box (510). Values shown in these boxes can be changed, and displayed after recalculating a coding potential. This operation can be done by pushing button (511).

In amino acid alignment indication pane (304), an alignment between amino acid sequences in amino acid sequence database is displayed as a segment. Details are described using FIG. 7. Setting cDNA sequence scale (601) as a coordinate, an alignment between amino acid sequences in amino acid sequence database is displayed as a segment. As an amino acid database, SWISS-PROT, OWL etc. are used. As described in the description in FIG. 3, an alignment obtained by analysis with TRANSQ or BLASTX is used. Herein, a case where TRANSQ is used is described. As described in FIG. 3, an alignment sorted with E-values obtained by BLASTX analysis performed prior to the comparison with TRANSQ is displayed as a segment. The alignment is arranged from top to bottom in ascending order according to E-value. First alignment (602) between a cDNA sequence and an amino acid sequence, second alignment (603) between those sequences, and third alignment (604) between those sequences are shown as examples of the thus obtained alignment. On the left side of alignments (602), (603) and (604), there is described value information (614) which characterizes each alignment (Identity, E-value of blastx analysis, length of an alignment (Al), length of non-aligned DNA side at 5′-end (NAb) and length of non-aligned amino acid side at 5′-end (NAa)). On the right side of alignments (602), (603) and (604), there are described information regarding an amino acid sequence (ID, definition etc.) A non-aligned region in each segment is displayed as segment (608). An aligned region is displayed in a distinctive pattern depending on the identity value of an alignment. This consistency level may be displayed with color. Segment (605) indicates the region corresponding to Identity≧90%, segment (606) indicates the region corresponding to 90%>Identity≧40%, and segment (607) indicates the region corresponding to 40%>Identity. The value of Identity is calculated in a window of a preset size, and the values of various regions of a sequence are calculated by sliding along the sequence. In an alignment between a cDNA sequence and an amino acid sequence, an insertion region on the DNA side (where insertion number is multiples of 3) is shown as segment (609), and a deletion region on the DNA side (where deletion number is multiples of 3) is shown as segment (610). With this, the information regarding a frame shift in an amino acid sequence obtained from an alignment can be confirmed concurrently for a plurality of alignments. Furthermore, it is possible to judge the significance of such insertions or deletions, depending on the identity region where the insertion or deletion of an alignment occurred. That is, where an insertion or deletion has occurred in a high identity region, significance is also high, on the other hand, where an insertion or deletion has occurred in a low identity region, the significance is also low. For example, the significance of insertion (609) on the DNA side, which locates at the same position on both alignments (602) and (603) is determined to be high, since identity at that position is more than 90%. On the other hand, the significance of deletion (610) on the DNA side, which locates on alignment (603) is determined to be low, since identity at that position is 40% or less. Moreover, in respect of alignment (604), since this cDNA shows an identity of 100% with a ribosomal protein, it can be assumed that the cDNA would constitute a chimeric gene with a ribosomal gene, and that the connection site would be at around 300 bases. It is possible for a user to judge a site where the editing of a cDNA sequence is conducted and an alignment to be used for editing by a total observation of insertion/deletion sites located on a plurality of alignments and identity of each site. Links to detailed information of each alignment is performed through check boxes (611), (612) and (613) located on the right side of an alignment segment. For example, by selecting check box (611), it becomes possible to display an amino acid sequence obtained by the selected first alignment as a segment on an amino acid indication frame described in FIG. 5. As described in connection with an amino acid indication frame, by concurrently comparing between the segment of an amino acid sequence obtained from an alignment and the segment of an ORF obtained from a cDNA sequence itself on 3 amino acid frames, the position where a frame shift has occurred and the transition of the frame shift become clear. Thus, confirming the existence and certainty of the frame shift in FIGS. 5 and 7, it is possible to edit the frame shift site of a cDNA sequence in an alignment. To link to the editing window of a cDNA sequence, an alignment to be edited is selected through check box (611), (612) or (613). Then, editing window (114) is generated by pushing editing button (111) shown in FIG. 2.

FIG. 8 shows details regarding an editing window. In an editing window, alignment (701) between a cDNA sequence and an amino acid sequence, as well as buttons (703), (704) and (705) for editing are displayed. In alignment (701) between a cDNA sequence and an amino acid sequence, the cDNA sequence and the translated amino acid sequence are text-displayed, being parallel to the known target amino acid sequence. A solid line between the translated amino acid sequence and the target amino acid sequence shows the consistency of the amino acids, and a colon and a full stop show the similarity level between amino acids, depending on the number of dots. In this alignment, it can be seen that the insertion of an a-base has occurred at position (702). That is to say, deeming the a-base to be an insertion base, it can be seen that the amino acid sequences around the a-base match well. Editing starts when a user directly deletes this a-base. The execution of editing result determination and post-analysis can be done by pushing a button for editing determination and post-analysis (703) marked as “Submit”. By pushing this button, cDNA sequence analysis and display process (106) is carried out again. The editing window can be closed by pushing the alignment between a cDNA sequence and an amino acid sequence and editing window close button (704) marked “Close”. The results edited before termination of editing and post-analysis can be reset by pushing a reset button for cDNA sequence editing (705) marked “Refresh”.

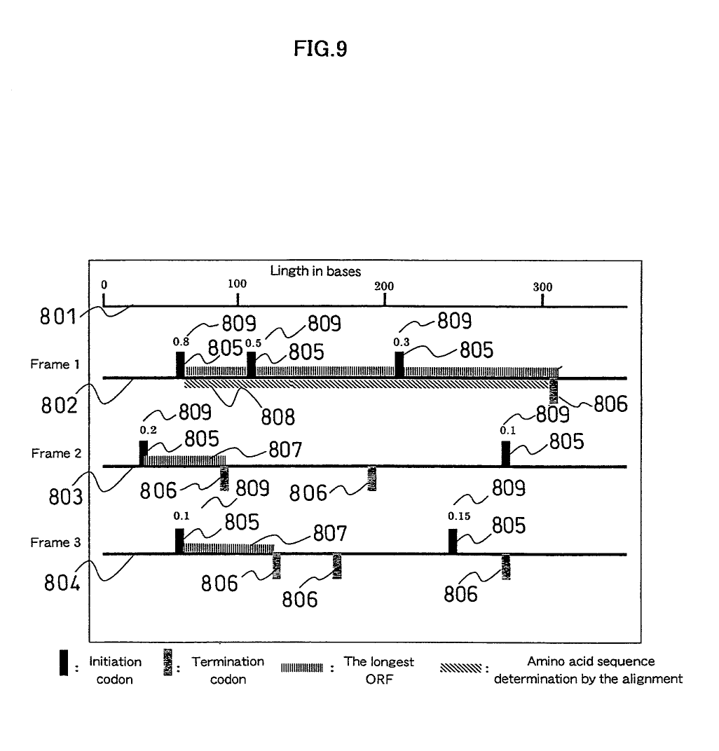

The results of the thus performed post-analysis after editing a cDNA sequence are immediately reflected in analysis results display window (108). FIG. 9 is an amino acid frame indication in respect of the results obtained by editing by deletion of an a-base, which was deemed to be an insertion base in FIG. 8. When compared with FIG. 5, it can be seen that the information on each frame is exchanged at a region at around 130 bases or more. That is, in FIG. 5, the segments on a frame of an amino acid sequence determined by an alignment are displayed astride frames 1 and 2, but in FIG. 9, the segments are integrated into a unified segment and displayed on only frame 1. From this result, validity of the editing of a cDNA sequence shown in FIG. 8 can be confirmed. Furthermore, it is shown that ATGpr value has been updated by the editing. The score value of ATG on the left side of frame 1 is significantly increased from 0.45 to 0.80, and this would be caused by the lengthening of the ORF initiating from ATG on the left side as a result of editing. Thus, the validity of editing can further be confirmed by the increase of ATGpr score value.

The present invention is not limited to the above-stated embodiments.

The present invention may be a computer-readable recording medium which records a program to allow a computer to function as the above-stated amino acid frame indication system, and may be any type of recording medium such as a magnetic tape, a CD-ROM, an IC card and a RAM card etc.

That is to say, the present invention may comprise a computer-readable recording medium which records a program to allow a computer to function as an amino acid frame indication system which comprises: input means for inputting a cDNA sequence; translation means for obtaining 3 amino acid frames translated by shifting one letter per frame along said input cDNA sequence; alignment means for generating an alignment between said input cDNA sequence and a DNA or amino acid sequence in a database to determine from the alignment an amino acid sequence translated from said input cDNA sequence on the basis of similarity information; and display means for displaying as a segment a region for the amino acid sequence determined by said alignment means on said 3 amino acid frames.

The present invention may further comprise a computer-readable recording medium on which is recorded a program which allows a computer to function as an amino acid frame indication system which comprises: input means for inputting a cDNA sequence; translation means for obtaining 3 amino acid frames translated by shifting one letter per frame along said input cDNA sequence; codon prediction means for predicting each of initiation and termination codons in said 3 amino acid frames; and display means for displaying an amount or symbol expressing the plausibility of an initiation codon at the initiation codon position as well as displaying the positions of said initiation and termination codons on said 3 amino acid frames.

The present invention may further comprise a computer-readable recording medium on which is recorded a program allowing a computer to function as an amino acid frame indication system which comprises: input means for inputting a cDNA sequence; translation means for obtaining 3 amino acid frames translated by shifting one letter per frame along said input cDNA sequence; codon prediction means for predicting each of initiation and termination codons in said 3 amino acid frames; coding potential calculation means for calculating coding potential showing coding region plausibility in each of said 3 amino acid frames; and display means for displaying the coding potential of said 3 amino acid frames on each frame or in another window, as well as displaying the positions of said initiation and termination codons on said 3 amino acid frames.

Effect of the Invention

According to the present invention, it becomes possible to effectively detect a frame shift by expressing, on each amino acid frame, the amino acid information of a cDNA sequence obtained by similarity comparison with the known amino acid sequences as well as the ORF display of an unknown cDNA sequence, and displaying the information (the plausibility of an initiation codon and coding potential graph) regarding an ORF statistically obtained at the same time, and possible to obtain a high precision amino acid sequence by editing the results.

What is claimed is:

1. An amino acid frame indication system which comprises: input means for inputting a cDNA sequence; translation means for obtaining 3 amino acid frames translated by shifting one letter per frame along said input cDNA sequence; alignment means for generating an alignment between said input cDNA sequence and a DNA or amino acid sequence in a database to determine from the alignment an amino acid sequence translated from said input cDNA sequence on the basis of similarity information; and display means for displaying as a segment a region of the amino acid sequence determined by said alignment means on said 3 amino acid frames.

2. The amino acid frame indication system according to claim 1, wherein said alignment means determines an amino acid sequence from an alignment between the amino acid sequence obtained by translating said input cDNA sequence by said translation means in 3 or 6 types of reading frames and an amino acid sequence in a database.

3. The amino acid frame indication system according to claim 1, wherein said alignment means determines an amino acid sequence, taking into consideration a codon gap in said input cDNA sequence.

4. The amino acid frame indication system according to claim 1, wherein said alignment means determines an amino acid sequence from an alignment between amino acid sequences translated from said input cDNA sequence and a DNA sequence in a database, while taking into consideration a codon gap between each of these DNA sequences.

5. The amino acid frame indication system according to claim 1, wherein said display means displays, as a segment, said generated alignment together with said three amino acid frames.

6. The amino acid frame indication system according to claim 5, wherein said display means displays an insertion or deletion position in a DNA sequence in said alignment displayed as a segment.

7. The amino acid frame indication system according to claim 5, wherein said display means displays, as a color, the local consistency of an alignment in said alignment displayed as a segment.

8. The amino acid frame indication system according to claim 1, wherein said display means displays, as text, an alignment between said generated cDNA sequence and a DNA or amino acid sequence in a database, together with said three amino acid frames.

9. An amino acid frame indication system which comprises: input means for inputting a cDNA sequence; translation means for obtaining 3 amino acid frames translated by shifting one letter per frame along said input cDNA sequence; codon prediction means for predicting each of initiation and termination codons in said 3 amino acid frames; and display means for displaying an amount or symbol expressing the plausibility of an initiation codon at the initiation codon position as well as displaying the positions of said initiation and termination codons on said 3 amino acid frames.

10. An amino acid frame indication system which comprises: input means for inputting a cDNA sequence; translation means for obtaining 3 amino acid frames translated by shifting one letter per frame along said input cDNA sequence; codon prediction means for predicting each of initiation and termination codons in said 3 amino acid frames; coding potential calculation means for calculating coding potential showing coding region plausibility in each of said 3 amino acid frames; and display means for displaying the coding potential of said 3 amino acid frames on each frame or in another window, as well as displaying the positions of said initiation and termination codons on said 3 amino acid frames.

11. The amino acid frame indication system according to claim 1 which comprises an editing means for editing said input cDNA sequence and resetting the edited cDNA sequence to said input cDNA sequence.

12. The amino acid frame indication system according to claim 11, wherein said editing means can perform editing while displaying the text of an alignment.

13. A method for amino acid frame indication which comprises: an input step for inputting a cDNA sequence; a translation step for obtaining 3 amino acid frames translated by shifting one letter per frame along said input cDNA sequence; an alignment step for generating an alignment between said input cDNA sequence and a DNA or amino acid sequence in a database to determine from the alignment an amino acid sequence translated from said input cDNA sequence on the basis of similarity information; and a display step for displaying as a segment a region of the amino acid sequence determined by said alignment step on said 3 amino acid frames.

14. A method for amino acid frame indication which comprises: an input step for inputting a cDNA sequence; a translation step for obtaining 3 amino acid frames translated by shifting one letter per frame along said input cDNA sequence; a codon prediction step for predicting each of initiation and termination codons in said 3 amino acid frames; and a display step for displaying an amount or symbol expressing the plausibility of an initiation codon at the initiation codon position as well as displaying the positions of said initiation and termination codons on said 3 amino acid frames.

15. A method for amino acid frame indication which comprises: an input step for inputting a cDNA sequence; a translation step for obtaining 3 amino acid frames translated by shifting one letter per frame along said input cDNA sequence; a codon prediction step for predicting each of initiation and termination codons in said 3 amino acid frames; a coding potential calculation step for calculating coding potentials showing coding region plausibility in each of said 3 amino acid frames; and a display step for displaying the coding potential of said 3 amino acid frames on each frame or in another window, as well as displaying the positions of said initiation and termination codons on said 3 amino acid frames.
